(19) United States
(12) Patent Application Publication
(10) Pub. No.: US 2015/0352309 A1
(43) Pub. Date: Dec. 10, 2015

(54) SINGLE USE BREATHING MASK WITH ADHESIVE GASKET

(71) Applicant: Periodic Breathing Foundation, LLC, East Providence, RI (US)

(72) Inventor: Robert W. Daly, Providence, RI (US)

(21) Appl. No.: 14/829,994

(22) Filed: Aug. 19, 2015

Related U.S. Application Data
(63) Continuation-in-part of application No. 13/911,122, filed on Jun. 6, 2013.

(51) Int. Cl. A61M 16/06

(52) U.S. Cl. CPC A61M 16/0688; A61M 2205/02

(57) ABSTRACT

A single use mask having an adhesive gasket is provided. The mask is of a two piece construction including a relatively hard attachment shell and a flexible gasket. The shell serves at the air chamber covering the mouth and nose of the patient as well as the point of attachment for the therapy tubing set. The flexible gasket and adhesive layer serves as the attachment means for affixing the mask to the patient's face. In contrast to the masks of the prior art, the gasket is formed from a sheet of foam material.

### CROSS-REFERENCE TO RELATED APPLICATIONS

This application is a continuation-in-part of U.S. patent application Ser. No. 13/911,122, filed Jun. 6, 2013 which claims priority from earlier filed U.S. Provisional Patent Application No. 61/656,241, filed Jun. 6, 2012.

### BACKGROUND OF THE INVENTION

The present invention relates to a breathing mask for use in connection with a therapeutic breathing apparatus. More specifically, the present invention relates to a mask and a method of forming a mask that allows improved adhesion to the patients face to facilitate improved sealing and performance.

There are a variety of medical conditions and environmental situations that require the use of a mask assembly. Such masks may be needed to protect a wearer's airways from the undesired effect of various substances that may be inhaled with the breathing air, or where breathing gases, to which medically indicated components are optionally added, are to be specifically introduced. This may be exemplified by breathing masks that are delivered with respirators in the broadest sense of the word. These include, among other things, devices for patients who require respiratory support for various reasons, e.g., sleep apnea or chronic obstructive pulmonary disease (COPD). Such disorders of spontaneous breathing are frequently treated with continuous positive airway pressure (CPAP) respirators or similar devices.

In using a CPAP device, a settable overpressure is made available through the use of a mask to support the patient's respiration. The supply frequently insures constant pressure over the entire breathing cycle via a breathing mask. An overpressure prevails in the breathing mask in relation to the environment, which is set between a few mbar and up to 50 mbar depending on the therapy. To maintain this overpressure in the interior of the mask, the mask is usually sealed with a seal between the mask body and the user's face. In many cases a leak rate of a few L per minute can be tolerated during such applications, in which fresh gas scavenging takes place in the interior of the mask. By contrast, however, maximum sealing action is desirable for other critical applications.

For example, in some cases medical clinicians, physical therapists and the like often very closely analyze the gas content of the exhaled breath of a person being medically tested or placed under some type of physical activity. Technology has developed in this field to a point where it is possible to analyze the gaseous percentage of exhaled breath of the person being tested and to quite closely analyze the various amount of the gasses components that are contained within each breath. In the past, when such tests were first run, the analytical techniques were unable to provide extremely accurate analysis of the gasses. Further, other conditions require that a patient's gasses be carefully monitored and controlled to treat their particular breathing disorder. Consequently, general trends were studied more than a specific analysis of each breath. With the improvements in the analytical techniques this has changed so that each breath can be carefully studied and the analytical techniques are sufficient to provide a very accurate analysis of the gasses components of each breath.

Because the analytical techniques have improved, the major problems associated with highly accurate analyses have switched from problems in chemical analysis to preventing the presence of dead spaces and/or leaks within the testing equipment. Consequently, there have been recent attempts in this industry to try to develop masks which highly conform to the face of the user and create comparatively very little dead space within the mask itself so there is little gas that collects between the user and the analytical equipment. One of the major problems with masks of this type is that the mask is made for a “standard face”. As can be readily determined by viewing a number of persons, face contours and overall shape of people vary substantially as well as does the relative size of the persons' head and consequently their face. Because of these variations it is not possible to make a mask that will fit every single person exactly. Even if a mask could be made to fit a person exactly, movement of various muscles in the face, such as during physical activity or breathing, may slightly disrupt the seal of the mask.

Some masks attempt to achieve a seal by pressing the mask against the face of the user to ensure the desired sealing. The wearing comfort of such masks is determined essentially by the manner in which the force applied to the mask is transmitted as a pressure, via the seal, onto the face in the area of the contact line between the mask and the face. Each area of the contact line must be pressed sufficiently firmly against the user's face especially in case of applications that operate with an overpressure in the interior of the mask in order to counteract the tendency of the mask lifting off. However, due to their nature, conventional mask bodies are only conditionally suitable for uniform transmission of forces to irregularly shaped and changing surfaces over the contour of their own edge, a pressing force that may lead to needlessly high pressing pressures at some points of the face must usually be preset in order to guarantee the desired sealing action. A reduction of these strong pressing forces by generally reducing the pressing force, which would be able to be set, for example, by means of the strap of the mask, would be very likely to lead to leakages in other areas of the contact line as a consequence.

Thus, the problem that continues to be present is that the mask must be pressed onto the face with a markedly stronger force than would be necessary to compensate the force that could enable the mask to be lifted off as a function of the internal pressure in the mask and the area on which this internal pressure acts. The pressing force is usually built up by a tension of the straps and is transmitted to the mask body. The higher the intended internal pressure and the more uniform the pressing pressure of the mask body on the face, the stronger must be the force with which the mask must be pressed on. If the internal pressure largely compensates the pressing pressure, the contact of the mask is not usually felt by the user to be unpleasant. However, sufficient sealing action cannot be expected in this state for the above-mentioned reasons in case of conventional masks.

In view of the above-described shortcomings of current mask technology, there exists a need in the art for a mask that can form a seal to a patients face while reducing complexity and the need for a variety of straps and harnesses. More specifically, there exists a need for a mask with an adhesive based seal that effectively seals to a patients face without inducing undue stress points on the seal or the patient's skin.

### BRIEF SUMMARY OF THE INVENTION

In this regard, the present invention provides a breathing mask and a method of making a breathing mask for use in connection with a therapeutic breathing apparatus. More specifically, the present invention relates to a mask and a method of forming a mask that allows improved adhesion to the patient's face to facilitate improved sealing and performance.

In one embodiment, the present invention is a layered mask construction, including a relatively hard attachment shell assembly and a flexible gasket. In contrast to the masks of the prior art, the gasket is formed from a sheet of foam material. The foam material is pre-molded before the gasket is die cut and attached to the mask shell. This step in the process is important as will be described in greater detail below.

The mask shell is configured to essentially cover the mouth and nose of the wearer and provides an attachment port for interconnectivity with a breathing apparatus as is known in the art. The shell may also be formed to include a peripheral portion and a removable insert where the breathing apparatus attaches. The removable insert allows for access to the patients mouth after the mask has been positioned and affixed for various reasons such as, for example, teeth brushing, administering of medications, vomit clean-up, etc. The gasket is adhered to the shell to create a contact/sealing interface for the user.

A first portion of the gasket is preformed in a molding or heat press process such that the contours are created within the foam material itself prior to laminating the assembly with the shell. This greatly reduces the stress introduced to the gasket at the various contours and direction changes. In this manner an adhesive is employed that allows placement, removal and re-adherence of the mask to the wearer without the leakage or adhesive strength issues

It is therefore an object of the present invention to provide a mask that can form a seal to a patients face while reducing complexity and the need for a variety of straps and harnesses. More specifically, it is an object of the present invention to provide a mask with an adhesive based seal that effectively seals to a patients face without inducing undue stress points on the seal or the patient's skin.

Further features and advantages of the invention, as well as structure and operation of various embodiments of the invention, are disclosed in detail below will reference to the accompanying drawings.

### BRIEF DESCRIPTION OF THE DRAWINGS

The present invention is described with reference to the accompanying drawings. In the drawings, like reference numbers indicate identical or functionally similar elements. Additionally, the left-most digit(s) of a reference number identifies the drawing in which the reference number first appears.

FIG. 1 is a front view of the breathing mask of the present invention;

FIG. 2 is a side view of the breathing mask of the present invention;

FIG. 3 is a rear view of the breathing mask of the present invention;

FIG. 3a is a rear view of a prior art breathing mask;

FIG. 4 is a cross-sectional view of the breathing mask of the present invention taken along line 4-4 of FIG. 1;

FIG. 4a is a cross-sectional view of a prior art breathing mask; and

FIG. 5 is a front view of the breathing mask of the present invention with a removable access point provided therein.

### DETAILED DESCRIPTION OF THE INVENTION

Now referring to the drawings, the breathing mask is shown and generally illustrated in the figures. It is notable that the mask of the present invention is described herein as a single use mask having an adhesive gasket for affixing the mask to the patient's face and is suitable in any context wherein the use of such a mask is indicated. Environments that may indicate use for the mask of the present invention include, but are not limited to, breathing disorder therapy relating to various sleep apnea conditions or Cheynne Stokes respiration, oxygen therapy and administration of anesthesia. The ultimate end result desired in the mask is that when applied to the user, the mask forms a seal and does not allow leakage of the patients exhaled breath or therapeutic gasses even when administered at normal therapeutic pressures ranging from 4 mm H₂O to as high as 20 mm H₂O.

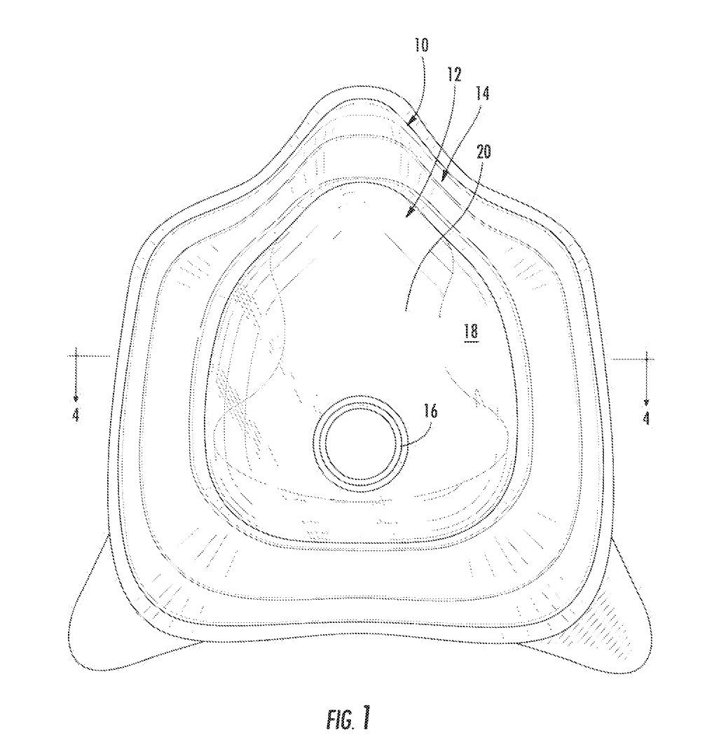

As seen at FIG. 1, most generally, the mask 10 of the present invention is of a two piece construction including a relatively hard attachment shell 12 and a flexible gasket 14. The shell 12 serves at the air chamber covering the mouth and nose of the patient as well as the point of attachment for the therapy tubing set. The flexible gasket 14 serves as the attachment means for affixing the mask 10 to the patient's face. In contrast to the masks of the prior art, the gasket is formed from a sheet of foam material. The foam material is pre-molded before or during the process wherein the gasket is die-cut and prior to attachment to the mask shell. This step in the process is important as will be described in greater detail below.

Further features and advantages of the invention, as well as the structure and operation of various embodiments of the invention, are described in detail below with reference to the accompanying drawings. The invention is not limited to the specific embodiments described herein. Such embodiments are presented herein for illustrative purposes only. Additional embodiments will be apparent to persons skilled in the relevant art(s) based on the teachings contained herein.

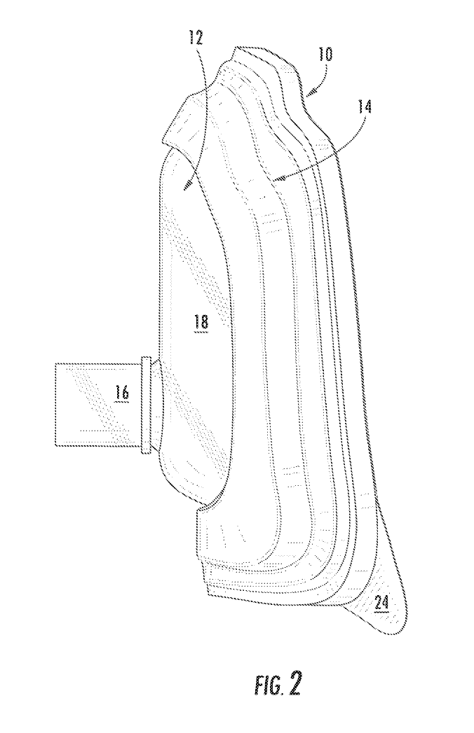

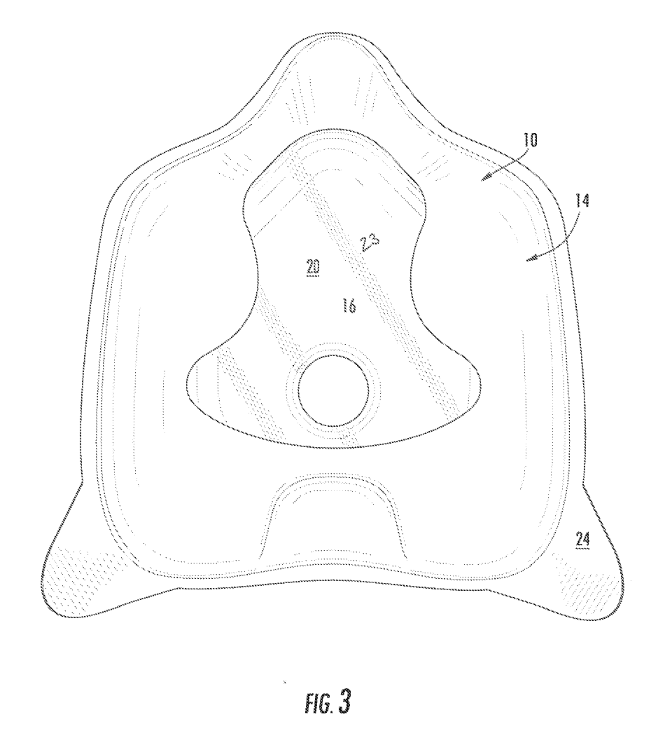

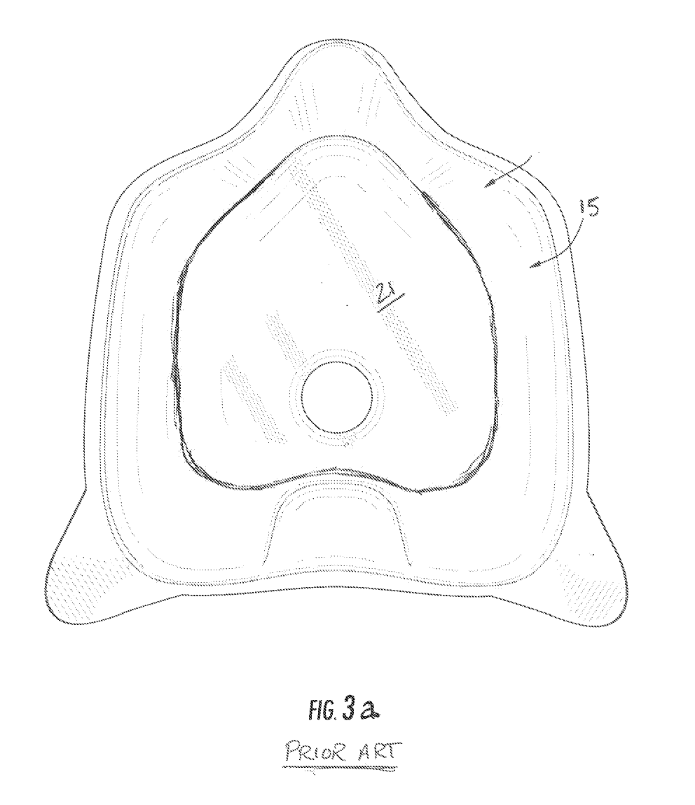

Turning now to FIGS. 1-3 to view the shell construction in detail, the mask shell 14 is configured to essentially cover the mouth and nose of the wearer and provides an attachment port 16 for interconnectivity with a breathing apparatus as is known in the art. The shell 14 itself is preferably formed in two layers, an outer layer 18 having the connection port 16 and an inner layer 20 that is received within the outer shell layer 18 having a reduced interior volume as compared to an interior volume of the outer shell layer 18. This is notable in comparison to the prior art mask shown at FIG. 3a wherein an interior volume 21 of the shell 15 is significantly larger than the interior volume 23 of the shell 14 of the mask 10 of the present invention.

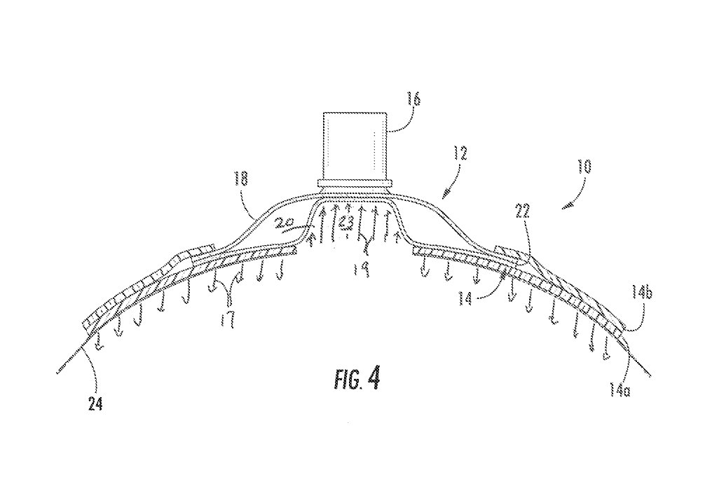

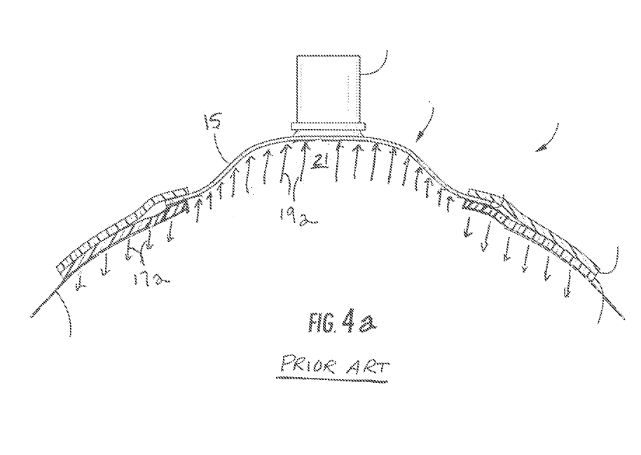

As can be seen in FIGS. 1-4, the inner shell layer 20 is contoured to fit more closely to the nose and mouth of the patient wearing the mask. This contouring of the inner shell 20 further serves to greatly reduce the interior volume 23 and more importantly the interior surface area of the inner shell 20. Still further, as best can be seen by comparing a cross-section of the mask of the present invention at FIG. 4 to the prior art mask at FIG. 4a, the reduction in volume 23 as compared to the prior art volume 21 also dramatically reduces the surface that is opposite the face of the patient, namely the surface subject to uplift forces. In the prior art, arrows 19a indicate a very large surface normal to the patients face that is subject to uplift force. In contrast, the double sheel arrangement of the present invention provides for a great reduction in surface area subject to uplift as indicated by arrows 19. When administering therapy at the known pressures in the art, the overall uplift force that displaces the mask away from the patient's face is the product of the pressure of the gasses applied to the connection port and the interior surface area of the shell that is opposite the face of the patient. As one skilled in the art will understand, pressures against the surfaces of the mask perpendicular to the face of the patient do not contribute to this uplift force as their vectors are in an outward, not upward direction. By creating an inner shell 20 with a smaller volume and normal projected surface area, the overall uplift force displacing the mask from the patient's face is reduced.

It is of further note that the adhesive gasket is adhered to the patients face as illustrated to the adhesive force resisting uplift at 17 while the prior art adhesive force is shown by arrows 17a. Clearly the surface area subject to adhesive force 17 in the present invention is much greater than the surface area subject to uplift force 19 while in the prior art the surface area subject to adhesive force 17a is much smaller than or about equal to the surface area subject to uplift force 19a.

Further, the shell 12 may be constructed as a single, low volume, inner shell using a heavier gauge plastic sheet material. In this embodiment, however, the shell tends to flex on the patient's face when breathing in and out. Accordingly, it is preferred that a lighter gauge polymer sheet material is employed and formed as a two layer shell to achieve lighter weight while enhancing structural stability and preventing the above noted flexing.

The inner and outer shell layers are preferably made using a vacuum molding process wherein a sheet of polymer is heated and drawn down over a mold to create the desired profiles.

The gasket 14 is preferably formed from two layers of foam material. In the prior art adhesive affixed masks, the bends in the gasket tended to introduce enough stress in the adhesives to prevent them from forming a reliable and acceptable seal with the patient. At the locations where the gasket was bent significantly, such as around the nose, the adhesive would release and form a leak. In the alternative, should the adhesive be strong enough to maintain the seal then removal of the adhesive and mask from the wearer becomes difficult and uncomfortable. The above noted problem is overcome in the present invention in that the gasket is pre-formed in a molding or heat press process such that the contours are created within the foam material itself prior to adhering it to the shell. This greatly reduces the stress introduced to the gasket at the various contours and direction changes. This pre-forming step is important in that it prevents the foam gasket from trying to return to its original flat shape thus allowing the gasket to remain adhered to the contours of a patient's face, such as over the bridge and into the creases of the patient's nose, without developing leaks.

The shell 12 has a flange 22 around its periphery that is trapped between two foam gasket layers 14a and 14b during manufacturing/assembly. There is difficulty in laminating foam to the polymer sheet materials used in forming the shell in a manner that achieves a quality and reliable seal. In accordance with the present invention, the preferred embodiment has the shell 12 disposed between two layers 14a, 14b of foam gasket 14. Once the first foam layer is heat formed as described above, the shell is placed therein and the second foam gasket having adhesive and a release liner 24 applied to a rear surface thereof is placed on top of the shell. The entire assembly is then heat pressed to fuse the two foam gasket layers to one another as well as to the flange of the shell. Further, an adhesive may be employed to assist in adhering the layers to one another.

It is an important feature of the present invention that the surface area of the gasket layer be greater than the surface area of the interior of the inner shell subject to uplift. Further still, it is important that the overall adhesive area of the inner gasket layer that is applied to the patient's face is greater than the surface area of the interior surface of the inner shell subject to uplift. In this manner an adhesive having less aggressive characteristics can be employed to adhere the mask to the patient's face thereby making the mask more comfortable to wear and easier to remove when therapy is completed. Preferably the surface area of the gasket is at least twice the surface area of the interior shell subject to uplift. More preferably the surface area of the gasket is three times the surface area of the interior shell subject to uplift. This allows a large area of adhesive having a lower adhesion to be employed in a manner that counteracts the uplift force of the therapy pressure described above.

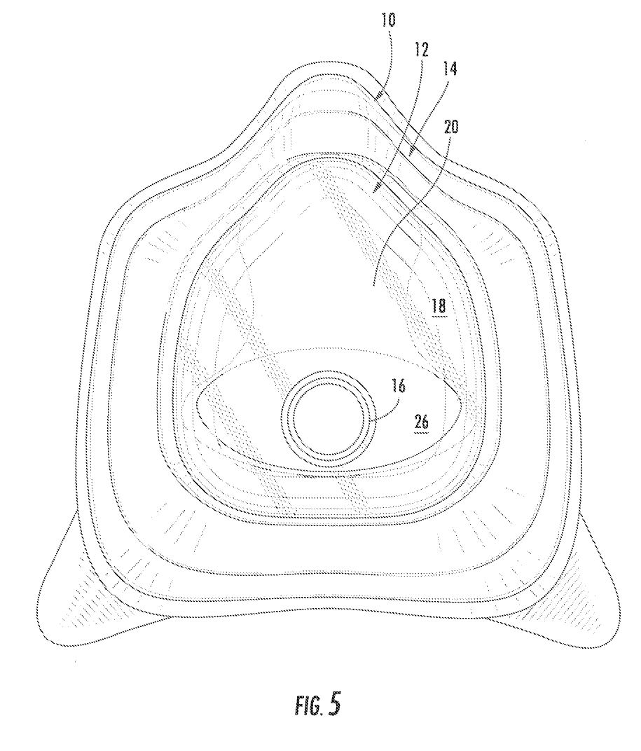

As can be seen at FIG. 5, the shell 12 can be formed to include a removable insert 26 where the breathing apparatus attaches. The removable insert 26 allows for access to the patients mouth after the mask has been positioned for various reasons such as, for example, teeth brushing, administering of medications, vomit clean-up, etc.

While the present invention is described herein with reference to illustrative embodiments for particular applications, the invention is not limited thereto. Those skilled in the art with access to the teachings provided herein will recognize additional modifications, applications, and embodiments within the scope thereof and additional fields in which the present invention would be of significant utility.

What is claimed:

1. A breathing mask comprising:
a shell, said shell having an attachment port for interfacing with a breathing apparatus, said shell having an interior volume and an interior surface area, a portion of said surface area subject to uplift created by said breathing apparatus; and
a gasket affixed to a periphery of said shell, said gasket having an adhesive on an interior surface thereof and an adhesive surface area, wherein said adhesive surface area is greater than said surface area subject to uplift.

2. The breathing mask of claim 1, wherein said adhesive surface area is at least twice said interior surface area subject to uplift.

3. The breathing mask of claim 1, wherein said adhesive surface area is at least three times said interior surface area subject to uplift.

4. The breathing mask of claim 1, said shell further comprising:
an outer shell layer; and
an inner shell layer, said inner shell having an interior volume and an interior surface area, a portion of said surface area subject to uplift, said attachment port extending through said inner and outer shell layers and in fluid communication with said interior volume, wherein said adhesive surface area is greater than said interior surface area subject to uplift.

5. The breathing mask of claim 4, wherein said adhesive surface area is at least twice said interior surface area subject to uplift of said inner shell layer.

6. The breathing mask of claim 4, wherein said adhesive surface area is at least three times said interior surface area subject to uplift of said inner shell layer.

7. The breathing mask of claim 4, wherein said outer shell layer and said inner shell layer cooperate to provide structural stability for said shell.

8. The breathing mask of claim 1, said gasket comprising:
a thermoformed foam material contoured to generally approximate a patient's facial contour.

9. The breathing mask of claim 1, said gasket comprising:
an outer layer;
an inner layer, wherein the flange of said shell is retained between said inner and outer layers; and
an adhesive layer applied to an inner surface of said inner layer.

10. The breathing mask of claim 1, wherein said outer and inner layers are a thermoformed foam material contoured to generally approximate a patient's facial contour.

11. A method of forming a breathing mask comprising:
forming a shell, said shell having an attachment port for interfacing with a breathing apparatus, said shell having an interior volume and an interior surface area subject to uplift; and
affixing a gasket to a periphery of said shell, said gasket having an adhesive on an interior surface thereof and an adhesive surface area, wherein said adhesive surface area is greater than said interior surface area subject to uplift.

12. The method of claim 11, wherein said adhesive surface area is at least twice said interior surface area subject to uplift.

13. The method of claim 11, wherein said adhesive surface area is at least three times said interior surface area subject to uplift.

14. The method of claim 11, the step of forming said shell further comprising:
forming an outer shell layer; and
forming an inner shell layer, said inner shell having an interior volume and an interior surface area subject to uplift;
placing said inner shell within said outer shell, said attachment port extending through said inner and outer shell layers and in fluid communication with said interior volume, wherein said adhesive surface area is greater than said interior surface area subject to uplift of said inner shell layer.

15. The method of claim 11, said gasket comprising:
a thermoformed foam material contoured to generally approximate a patient's facial contour.

16. The method of claim 11, said gasket comprising:
an outer layer;
an inner layer, wherein the flange of said shell is retained between said inner and outer layers; and
an adhesive layer applied to an inner surface of said inner layer.
